(19) United States
(12) Patent Application Publication
(10) Pub. No.: US 2014/0335210 A1
(43) Pub. Date: Nov. 13, 2014

(54) METHOD AND AGROCHEMICAL COMPOSITION FOR USING LARCH WOOD EXTRACTS IN AGRICULTURE

(71) Applicants: Sergey V. Philippov, Moscow (RU); Igor M. Bogorodov, Moscow (RU)

(72) Inventors: Sergey V. Philippov, Moscow (RU); Igor M. Bogorodov, Moscow (RU)

(73) Assignee: FLAVITPURE, INC., Cheyenne, WY (US)

(21) Appl. No.: 13/892,986

(22) Filed: May 13, 2013

(51) Int. Cl. A61K 36/15; A61K 31/352

(52) U.S. Cl. CPC A61K 36/15; A61K 31/352
USPC 424/770

(57) ABSTRACT

A method of using larch wood extracts as natural compounds to give superior resistance to plants, plant parts, fruits and/or flowers against pathogens as bacteria and fungi. The method includes using larch wood extracts as natural compounds that naturally protect plant tissues against ultraviolet radiation and temperature, thus giving protection against sunburn to plants, plant parts, fruits and/or flowers during their development. Additionally, the method includes using larch wood extracts as natural compounds to naturally increase adaptive potential of the seedlings, increase the root growth of seedlings, and to maximize the ability of plants to grow vigorously in response to changing environmental conditions. The above natural compounds are derived from a plant material and are antioxidant Dihydroquercetin (taxifolin), polysaccharide Arabinogalactan, a combination of Arabinogalactan with Dihydroquercetin (taxifolin) and wood oleoresin including oil and resin, wherein all these natural compounds are extracted from larch wood and\or by-products of logging industry.

### REFERENCES

[1]. Fitzpatrick A., Roberts A and Witherly S. (2004). Larch Arabinogalactan: a novel and multifunctional natural product. Agro Food industry Hi-Tech 15(1):30-32.
[2]. Ohr L. M. Arabinogalactan Adds More than Health Benefits//Prepared Foods. 2001. V. 170. No. 1. Page 55.
[3]. Potapovich A I, Kostyuk V A. Comparative study of antioxidant properties and cytoprotective activity of flavonoids. Biochemistry (Mosc.). 2003 May; 68(5):514-9.
[4]. J. MIZICKO, C. H. LIVINGSTON AND G. JOHNSON. (1973). THE EFFECTS OF DIHYDROQUERCETIN ON THE CUT SURFACE OF SEED POTATOES. AMERICAN POTATO JOURNAL. Vol. 51, pp. 216-218.
[5]. Tamara I. Balakhnina, Anatoly B. Gavrilov. Teresa M. Wlodarczyk. AnetaBorkowska, Magdalena Nosalewicz and Irina R. Fomina. (2009). Dihydroquercetin protects barley seeds against mold and increases seedling adaptive potential under soil flooding. Plant Growth Regulation, Volume 57, Number 2, pages 127-135.
[6]. Greimel, A.; Koch, H. (1977). Biochemie und Physiologie der Pflanzen (BPP) 171(5): 419-424.
[7]. V. I. Bol'shakova. V. A. Khan. Zh. V. Dubovenko, E. N. Shmidt, and V. A. Pentegova. TERPENOLDS OF THE OLEORESIN OF THE LARCH GROWING IN KAMCHATKA. Novosibirsk Institute of Organic Chemistry, Siberian Branch, Academy of Sciences of the USSR. Translated from Khimiya Prirodnykh Soedinenii. No. 3, pp. 340-343, May-June, 1980. © 1980 Plenum Publishing Corporation.
[8]. G. F. Chernenko and E. N. Shmidt. NEIUTRAL EXTRACTIVE SUBSTANCES FROM THE BARK OF Larixsibirica. Novosibirsk Institute of Organic Chemistry, Siberian Branch, Academy of Sciences of the USSR. Translated from Khimiya Prirodnykh Soedinenii, No. 6, pp. 833-834, November-December. 1990. © 1991 Plenum Publishing Corporation.
[9]. L. G. D'yachenko, V. I. Roshchin, and V. E. Kovalev. NEUTRAL COMPOUNDS OF THE EXTRACTIVE SUBSTANCES OF Larixgmelini. S. M. Kirov Academy of Wood Technology, Leningrad. Translated from Khimiya Prirodnykh Soedinenii, pp. 56-63, January-February, 1986. © 1986 Plenum Publishing Corporation.
[10]. Lisina, A. I., L. N. Volskil. Y. G. Leont'eva and V. A. Pentegova. (1969). Extractives of the heartwood and sapwood of Larixgmelini. Izu. Sib. Otd. Akad. Nauk SSSR. Ser. Khim. Nauk. 6:10.2-104 (not seen. Chem. Abstr. 72; 68404b).
[11]. Kara'csonyi S, Kova'cik V. Alfoldi J, Kubackova M (1984) Chemical and 13C-N.M.R. studies of an arabinogalactan from Larixsibirica L. Carbohydr Res 134:265-274. Odonmazig, P. Ebringerova. A. Machova, E. Alföldi, J. (1994) Structural and molecular properties of arabinogalactan isolated from Mongolian larchwood (Larixdahurica L.). Carbohydr. Res. 252: 317-324.
[12]. Tatjana Stevanovic. Papa Niokhor Diouf and Martha Estrella Garcia-Perez (2009). Bioactive Polyphenols from Healthy Diets and Forest Biomass. Current Nutrition & Food Science, Vol. 5, No. 4, p. 264-295.
[13] Schijlen E G W, de Vos C H R, van Tunen A J, Bovy A G. (2004) Modification of flavonoid biosynthesis in crop plants. Phytochemistry, 65: 2631-48.
[14] Tyukavkina, N. A., Lapteva. K. I., Larina V. A. (1967). Extractives of Larixdahurica. Quantitative content of quercetin and dihydroquercetin. Chemistry of Natural Substances. Issue 5, pages 298-301.
[15]. E. E. Nifant'ev, M. P. Koroteev. G. Z. Kaziev, A. A. Uminskii, A. A. Grachev, V. M. Men'shov, Yu. E. Tsvetkov, N. E. Nifant'ev, V. K. Bel'skii. A. I. Stash. (2006). On the Problem of Identification of the Dihydroquercetin Flavonoid. ISSN 1070-3632, Russian Journal of General Chemistry, 2006. Vol. 76, No. 1, pp. 161-163. Pleiades Publishing. Inc., 2006. Original Russian Text published in Zhurnal Obshchei Khimii, 2006, Vol. 76, No. 1, pp. 164-166.
[16]. KOES. R. E.: QUATTROCCHIO. F.; Mol. J. N. M. The flavonoid biosynthetic pathway in plants: Function and evolution. Bio Essays, v. 16, p. 123-131, 1994.
[17]. Dixon, R. A. and Paiva, N. L. (1995). Stress-Induced Phenylpropanoid Metabolism. Plant Cell. No. 7(7): pp. 1085-1097.
[18]. Winkel-Shirley B. (2002). Biosynthesis of flavonoids and effects of stress. Curr. Opin. Plant. Biol. No. 5(3), pp. 218-23.
[19]. Rudman, P. 1963. The causes of natural durability in timber. Part XI. Some tests on the fungi toxicity of wood extractives and related compounds. Holzforschung 17 (21): 54-57.
[20]. Eaton, R. A. and Hale, M. D. C. 1993. Natural durability. 311-318 pp in Hall, C., eds. Wood: Decay, pests and protection, vol. 1. Chapman & Hall, London.
[21]. Langenheim, J. H. (1994). Higher-plant terpenoids: a phytocentric overview of their ecological roles. J ChemEcol 20:1223-1280.
[22]. Trombetta, D., Castelli, F., Sarpietro. M., Venuti, V., Cristani, M., Daniele, C., et al. (2005). Mechanisms of Antibacterial Action of Three Monoterpenes. Antimicrobial Agents and Chemotherapy, 49(6), 2474-2478.
[23]. Inouye, S., Takizawa, T., & Yamaguchi, H. (2001). Antibacterial activity of essential oils and their major constituents against respiratory tract pathogens by gaseous contact. Journal of Antimicrobial Chemotherapy, 47, 565-573.
[24]. Hammer, K., Carson, C., & Riley. T. (2003). Antifungal activity of the components of Melaleucaalternifolia (tea tree) oil. Journal of Antimicrobial Chemotherapy, 53, 853-860.
[25]. Özçlik, B., Gürbtiz. I., Karaoglu, T., & Yesilada, E. (2009). Antiviral and antimicrobial activities of three sesquiterpene lactones from Centaureasolstitialis L. ssp. solstitialis. Microbiology Research. 164(5), 545-552.
[26]. Raju, G., & Sanjay. M. (2009). Recent developments in anti-inflammatory natural products. Medicinal Research Reviews, 5, 767-20.
[27]. Sivropoulou, A., Papanikolaou. E., Nikolaou, C., Kokkini, S., Lanaras, T., &Arsenakis, M. (1996). Antimicrobial and Cytotoxic Activities of Origanum Essential Oils. Journal of Agriculture and Food Chemistry, 44(5), 1202-1205.
[28]. Ogura, K. (1998) in Comprehensive Natural Products Chemistry: Isoprenoid Biosynthesis, ed. Cane, D. E. (Pergamon, Oxford), in press.
[29]. Wise, M. L. &Croteau, R. (1998) in Comprehensive Natural Products Chemistry: Isoprenoid Biosynthesis, ed. Cane. I). E. (Pergamon. Oxford), in press.
[30]. Cane, D. E. (1998) in Comprehensive Natural Products Chemistry: Isoprenoid Biosynthesis, ed. Cane. D. E. (Pergamon, Oxford), in press.
[31]. MacMillan, J. (1998) in Comprehensive Natural Products Chemistry: Isoprenoid Biosynthesis, ed. Cane, D. E. (Pergamon, Oxford), in press.
[32]. Connolly. J. D. & Hill, R. A. (1991) Dictionary of Terpenoids (Chapman & Hall, London).
[33]. Patent No. US 2007/0232495 A1. Nappa et al. COMPOSITIONS AND METHODS TO ADD VALUE TO PLANT PRODUCTS, INCREASING THE COMMERCIAL QUALITY, RESISTANCE TO EXTERNAL FACTORS AND) POLYPHENOL CONTENT THEREOF.
[34]. WO/1998/059056 VON WETTSTEIN et al. PATHOGEN-RESISTANT MONOCOT PLANTS AND METHOD
[35]. Snyder B. A. and Nicholson R. L. (1990), Synthesis of phytoalexins in Sorghum as a site-specific response to fungal ingress. Science 248, pp. 1637-1639.
[36]. Feucht W., Treutter D., and Christ E. (1996). Shot hole disease in sour cherry: defensive flavanol barriers. J. Plant Dis. Protect. 103, pp. 279-287.
[37]. Jonathan J. Turnbull et al. (2004). Mechanistic Studies on Three 2-Oxoglutarate-dependent Oxygenases of Flavonoid Biosynthesis. THE JOURNAL OF BIOLOGICAL CHEMISTRY, Vol. 279, No. 2, pp. 1206-1216.
[38]. Bovy, A., et al. Plant Cell 2002; 14:2509-2526. Scheme of the Flavonoid Biosynthesis Pathway.
[39]. Jende-Strid B. Genetic control of flavonoid biosynthesis in barley//Hereditas. 1993. V. 119. P. 187-204.
[40]. Kristiansen K N. Rohde W. (1991). Structure of the Hordeumvulgare gene encoding dihydroflavonol-4-reductase and molecular analysis of ant18 mutants blocked in flavonoid synthesis. Molecular and General Genetics, No. 230, pp. 49-59.
[41]. BarbroJende-Strid. (1985). Phenolic acids in grains of wild-type barley and proanthocyanidin-free mutants. CARLSBERG RESEARCH COMMUNICATIONS. Volume 50, Number 1, pp. 1-14.
[42]. Tamara I. Balakhnina, Anatoly B. Gavrilov, Teresa M. Wlodarczyk, AnetaBorkowska, Magdalena Nosalewicz and Irina R. Fomina. (2009). Dihydroquercetin protects barley seeds against mold and increases seedling adaptive potential under soil flooding. Plant Growth Regulation, Volume 57, Number 2, pages 127-135.
[43]. Sanzani S. M., De Girolamo A., Schena L., Solfrizzo M., Ippolito A., 2007. Effect of quercetindihydrate and umbelliferone postharvest treatments on blue mold and patulin accumulation in apples. COST action 924—Novel approaches for the control of postharvest diseases and disorders. Bologna, Italy 3-5 May 2007 (in press).
[44]. J. MIZICKO, C. H. LIVINGSTON AND G. JOHNSON. (1973). THE EFFECTS OF DIHYDROQUERCETIN ON THE CUT SURFACE OF SEED POTATOES. AMERICAN POTATO JOURNAL, Vol. 51, pp. 216-218.
[45]. Greimel, A.; Koch, H. (1977). Biochemie und Physiologie der Pflanzen (BPP) 171(5): 419-424.
[46]. Winkel-Shirley B: Flavonoid biosynthesis: a colorful model for genetics, biochemistry, cell biology and biotechnology. Plant Physiol 2001, 126:485-493.
[47]. Landry L G, Chapple C C S, Last R L: Arabidopsis mutants lacking phenolic sunscreens exhibit enhanced ultraviolet-B injury and oxidative damage. Plant Physiol 1995, 109:1159-1166.
[48]. Fabienne Dédaldéchamp and Christiane Uhel. (1999). Induction of anthocyanin synthesis in non pigmented grape cell suspensions by acting on DFR substrate availability or precursors level.
[49]. Bertrand Garcia-Moreno. (2009). Adaptations of proteins to cellular and subcellular pH. Journal of Biology, 8:98.
[50]. V. N. Nurminsky. N. V. Ozolina, J. G. Sapega A. O. Zheleznykh, E. V. Pradedova, A. M. Korzun, and R. K. Salyaev. (2009). The Effect of Dihydroquercetin on Active and Passive Ion Transport Systems in Plant Vacuolar Membrane. Biology Bulletin. Vol. 36, No. 1, pp. 1-5. © Pleiades Publishing, Inc., 2009. ISSN 1062-3590.
[51]. MARK JACOBS and PHILIP H. RUBERY Science 15 Jul. 1988: Vol. 241, no. 4863, pp. 346-349.
[52]. Effects of Flavonoids on the Polar Transport of Auxins Physiologia Plantarum Volume 38 Issue 4, Pages 262-266 Published Online: 28 Apr. 2006.
[53]. Greimel, A.; Koch. H. 1976 Biochemie und Physiologie der Ptlanzen 171(5): 419-423.
[54]. Withers. J. C., Shipp. M. J., Dodd, A. J., and Wyatt, S. E. (In Review) GRAVITY PERSISTENT SIGNAL 1 reveals two novel cytochrome P450s involved in plant gravitropism. Plant Physiology.
[55]. Journal “Doklady N A S Belarus” 2008. Saskevitch P. A., Tsyganov A. R., Gurikova E. I. Efficiency of application of natural growth regulators when cultivating spring rape, pp. 116-119.
[56]. Chekurov, V. S., Sergeeva, S. I., Zhalieva, L. D., Kozlov, V. E., and Vakulenko, V. V., Zashchita Karantin Rast., 2003, no. 9, pp. 20-21.
[57]. Phillips. M. A. and Croteau, R., Trends Plant Sci., 1999, vol. 4, no. 5, pp. 184-190.
[58]. Trapp, S. and Croteau, R., Ann. Rev. Plant Physiol. Plant. Mol. Biol., 2001, vol. 52, pp. 689-724.
[59]. Raldugin. V. A., Zh. Ros. Khim. Obshch. im. D. I. Mendeleeva, 2004, vol. 48, no. 3, pp. 84-88.
[60]. Norin, T1., Phytochemistry, 1972, vol. 11, no. 4, pp. 1231-1242.
[61]. Maria Jose Abad et al. (2007). Active antifungal substances from natural sources. ARKIVOC (vii) pp. 116-145.
[62]. Chappel, J., Ann. Rev. Plant Physiol. Plant. Mol. Biol., 1995, vol. 46, pp. 521-547.
[63]. V. I. Bol'shakova, L. I. Demenkova. N. Shmidt, and V. A. Pentegova Novosibirsk Institute of Organic Chemistry, Siberian Branch. Academy of Sciences of the USSR. Translated from Khimiya Prirodynkh Soedinenii, No. 6, pp. 812-816, November-December, 1988.
[64]. TERPENOIDS OF THE OLEORESIN OF Larixolgensis. Larix Gmelinii (Rupr.) and Larimcajanderi Mayr. Khimiya Prlrodnykh Soedinenii, No. 1, p. 110. January-February, 1983. V. A. Khan, V. I. Bolshakova, E. N. Shmidt. Zh. V. Dubovenko, and V. A. Pentegova.
[65]. Wagner et al., 1983; Schuh and Benjamin, 1984; Rose et al., 1981.
[66]. Terziev. N. (2002b): Properties and Processing of Larch Timber—a Review based on Soviet and Russian Literature.

### FIELD OF THE INVENTION

This invention relates to the use of larch wood extracts as natural compounds that give more resistance to plants, plant parts, fruits and/or flowers against pathogens as bacteria and fungi. The invention is also related to the use of larch wood extracts as natural compounds that naturally protect plant tissues against ultraviolet radiation and temperature, thus giving protection against sunburn to plants, plant parts, fruits and/or flowers during their development. Additionally, the present invention is related to the use of larch wood extracts as natural compounds that naturally increase adaptive potential of the seedlings, increase the root growth of seedlings, to maximize the ability of plants to grow vigorously in response to changing environmental conditions. Finally, the present invention is related to plants, plant parts, fruits, flowers and/or propagating material treated with the larch wood extracts described in the present document. The present invention is directed to the use of wood extracts or natural compounds in particular antioxidant Dihydroquercetin (taxifolin), polysaccharide Larch Arabinogalactan. Larch Arabinogalactan combining with Dihydroquercetin (taxifolin) and wood oleoresin comprising oil and resin for applications in agriculture, wherein wood extracts are suggested to use as natural compounds for exposing numerous benefits within commercial agricultural purposes, wherein agriculture generally speaking refers to human activities also called farming or husbandry is the cultivation of plants, plant parts, fruits and/or flowers and other life forms for food, fiber, bio-fuel and other products used to sustain life.

### BACKGROUND OF THE INVENTION

Wood extracts have been applied in medicine, pharmacy and skin care since the ancient times. The present invention is primarily focused on the exploitation of wood extracts from residues of Conifer wood species. At present these residues are mainly used as a fuel material. The material is cheap and easily available in high amounts. As this material comprises waste products in wood industry, the exploitation of wood extracts from this material for agricultural purposes would significantly enhance its value. Conifer wood species, especially those from the family of Pinaceae are considered rich sources of above mentioned natural compounds. The emphasis is put on residues of wood transformation such as bark, butt logs, roots and knot wood as these materials represent particularly rich resources for flavonoids, particularly Dihydroquercetin (taxifolin), polysaccharides, particularly Larch Arabinogalactan and Larch Arabinogalactan combined with Dihydroquercetin (taxifolin) and wood oleoresin, particularly oil and resin, wherein such natural compounds are extracted or isolated from plant genus Larix, especially from the Larixcajanderi, Larixczekanowskii, Larixdahurica, Larixgmelinii, Larixkamtschatica, Larixrussica, Larixsibirica, Larixsukaczewii.

The plant genus Larix refers generally to any of the numerous conifers in the family of Pinaceae that have deciduous needlelike leaves. Larch wood is known to contain lignans, flavonoids, polysaccharides and oleoresin. Applications for larch wood extracts, in particular the polysaccharide Arabinogalactan and flavonoid Dihydroquercetin (taxifolin), are found in the food, pharmaceutical and cosmetic industries. Larch arabinogalactan, a water-soluble polysaccharide deriving mainly from plant genus Larix, is the source of dietary fiber, but has also confirmed effects as prebiotic [1,2]. The flavonoid constituents of larch wood in particular the flavonoid Dihydroquercetin (taxifolin) is known to possess good antioxidant, anti-fungi, mold and plant growth stimulator activities [3,4,5,6]. The oleoresins of the coniferous trees are well known in the flavor, fragrance, cosmetic and pharmaceutical industries. At the present time, the oleoresins of various species of larch have been studied in detail to consider actual content of biological active natural compounds [7,8,9]. There is information on the composition of the oleoresin terpenoids as extractive substances of the trunk part of the European larch and the heart and sapwood and the bark of the Larixcaianderi, Larixczekanowskii, Larixdahurica, Larixgmelinii, Larixkamischatica, Larixrussica, Larixsibirica, Larixsukaczewii. The neutral substances composing 50% of the weight of the larch oleoresin were represented by hydrocarbons and oxygen-containing compounds deterpenes (16 and 34%, respectively). Hydrocarbons are presented by monoterpene hydrocarbons, sesquiterpenes and diterpene hydrocarbons and aldehydes. The main components of the neutral substances are bicyclic compounds deterpenes or deterpenoids with the labdane structure: epimanool (˜15%), and larixol (˜40%) and its monoacetate (larixylacetate ˜28%), making up about one-third of the neutral substances. In the acidic fraction of the oleoresin, isopimaric acid (40%) predominates [10]. Diterpenoids are constituents of natural resins, such as colophony resin, which is gained from conifer trees like spruce, firs and pines. Larches belong to the family Pinaceae, as already mentioned.

According to the present invention “wood extracts” refers to all kind of extractable raw wood material obtained from a tree of the genus Larix. Preferably the wood extracts are obtained from extractable raw wood material used from Larixcajanderi, Larixczekanowskii, Larixdahurica, Larixgmelinii, Larixkamtschatica, Larixrussica, Larixsibirica, Larixsukaczewii. The larch wood material can, however, derive from other members of the genus Larix as well. Preferably the extractable larch wood material is larch sawdust, which is a waste product in wood industry. It is inexpensive and easily available in high amounts. The term “larch sawdust” also refers to larch wood shavings. Other kinds of waste wood from larch (e.g. bark, wastes accruing in woodcutting, scrap wood) can also be used within the frame of the present invention.

As used herein, the art-recognized term “Larch Arabinogalactan” includes the plant derived class of long, densely branched low and high-molecular polysaccharides MW: 3,000-120,000. Larch Arabinogalactan consists of a main chain of b-D-(1fi3)-galactopyranose units (b-D-(1fi3)-Galp) where most of the main-chain units carry a side chain on C-6 [fi3,6)-Galp-(1fi]. Almost half of these side chains are b-D-(1fi6)-Galp dimers, and about a quarter are single Galp units [FIG. 2,3]. The rest contain three or more units. Arabinose is present both in the pyranose (Arap) and furanose (Araf) forms, attached to the side chains as arabinobiosyl groups [b-L-Arap-(1fi3)-LAraf-(1fi] or as terminal a-L-Araf e.g. a single L-arabinofuranose unit or 3-O-(β-L-arabinopyranosyl)-α-L-arabinofuranosyl units [11].

Terms “emulsifier” and “emulsifying compound” include a compound which comprises surface-active molecules and which can stabilize a mixture or dispersion of otherwise immiscible compounds or liquids (e. g., an emulsion). Generally emulsifiers act either by coating one or more of the components of the mixture to prevent coalescing and/or alter the surface tension at the interface of suspended droplets. “Organic emulsifiers” include emulsifier molecules which may be identified and/or produced from an organism (e. g., plant material, animal material). In the present invention, the preferred emulsifiers are extracts from the Larch tree species particularly Larch Arabinogalactan and Larch Arabinogalactanthat contains Dihydroquercetin (taxifolin), generally about 5% to 30% Dihydroquercetin (taxifolin). Emulsification properties are obtainable with at a little as 3% Dihydroquercetin (taxifolin), however, formulations comprising between 5-12% are preferred.

Moreover, improvements to existing methods and compositions are encompassed in the invention described herein, at least in part, by virtue of the fact that the emulsifying agent Dihydroquercetin (taxifolin) and emulsifier Larch Arabinogalactan itself, are natural products. As such, they provide novel and improved means of using aqueous-recalcitrant compounds with concurrently diminished hazards and concerns associated with synthetic emulsifiers or concentrating compounds.

As used herein, the art-recognized term “Dihydroquercetin (taxifolin)” includes the plant derived flavonoid, comprising generally which relates to dihydroflavonol subclass of flavonoid family, the derivatives of phenylpropanoid metabolism. Their structures are based on C6-C3-C6 skeletons [FIG. 1], the A ring of the flavonoid structure being acetate derived (3×C2) and the C and B rings originating from cinnamic acid derivatives (phenylpropanoid pathway). “Dihydroquercetin (taxifolin)” is the compound having molecule structure is based on C6-C3-C6 skeleton consisting of two aromatic rings joined by a three carbon link with the absence of the C2-C3 double bond and have two chiral carbon atoms in position 2 and 3. The A ring of the flavonoid structure being acetate derived (3×C2) and the C and B rings originating from cinnamic acid derivatives (phenylpropanoid pathway). Consequently, the B-ring can be either in the (2S)- or (2R)-configuration. The C-3 atom of dihydrotlavonolDihydroquercetin (taxifolin) bears both a hydrogen atom and a hydroxyl group, and is therefore an additional center of asymmetry. Thus, four stereoisomers are possible for each dihydroflavonol structure. (2R,3R), (2R,3S), (2S,3R), and (2S,3S). All four configurations have been found in naturally occurring dihydroflavonols, but the (2R,3R)-configuration is by far the most common[12,13,14].

Term “terpene” includes hydrocarbon compounds which may be of biological origin and which have carbon skeletons formally derived from isoprene(IP) (CH2=C(CH3) CHCH2). The terpene compounds of the invention include (i) monoterpenes (C10=2 IP units), (ii) sesquiterpenes(C15=3 IP units), (iii) diterpenes (C20=4 IP units) including their acidic derivatives as major constituents of resins, (iv) sesterpenes (C25=5 IP units), (v) triterpenes (C30=6 IP units) of varying structures and omnipresent in vascular plants, (vi) tetraterpeniccarotenoids (C40=8 IP units) abundant in our food intake. The term “terpenoids” is used to describe the oxygenated derivatives of those hydrocarbons.

Flavonoids are involved in a vast array of different biological functions in plants. They play a crucial role in the symbiotic plant-microbe interactions (nodule formation of nitrogen fixing bacteria in leguminous plants) and in plant sexual reproduction by promoting the pollen tube development [16]. Flavonoids also have apparent roles in plant stress defense, such as in protection against damage caused by pathogen attack, in wounding or in excess of UV light. The low availability of nitrogen or phosphorus, and low temperatures affect flavonoid levels in plants [17.18]. The localization of flavonoids in the epidermal layers of plants and their known ultraviolet absorptive properties has led to a suggestion that they can serve as shields against potentially harmful radiation. There is a growing body of evidence for the role of flavonoids in photo protection [18]. There is a strong association between flavonoid biosynthesis and plant stress, which can cause flavonoid levels to increase in vegetative shoots and roots. There is evidence that flavonoids offer protection against many of these stressors e.g. second line of defense. On the role of Dihydroquercetin (taxifolin) in high plants the attention was paid quite long time ago due its properties to extend durability of trees where Dihydroquercetin (taxifolin) was found. Free hydroxyl groups are essential if phenolic compound, especially Dihydroquercetin (taxifolin), which acts as uncoupling agent that inhibits oxidative phosphorylation, the main source of energy in decay fungi [19]. Dihydroquercetin (taxifolin) is a molecule mainly found in species of the genus Larix, Cedrus and Pseudotsuga [20]. Dihydroquercetin (taxifolin) has been described as an antifungal agent; yet, dihydroquercetin (taxifolin) is mainly responsible for the high durability of the species mentioned above [19].

As a member of the pine family, the larch produces a resinous sap, called arabinogalactan, that gives the wood a water-resistant property. The wood is therefore sinewy and strong. Unlike many deciduous trees and some conifers, the larch tolerates very cold temperatures of at least −58° F. (−50° C.), and so is available to loggers in some the most remote areas of the boreal forests. Since the arabinogalactan is a complex sugar, it protects the tree from injury during freeze-thaw cycles, as well as damage from lightning strikes. Larch Arabinogalactan is used as an emulsifier, stabilizer, binder or bonding agent in essential oils, humectants, non-nutritive sweetener, flavor base, processing aid and stabilizer and considered to be bioavailability-enhancing and surface active agent, which can function as surfactant, emulsifier, foam modulator, and/or active ingredient dispersion agent. Larch Arabinogalactan is highly branched it is not subject to viscosity problems, as compared to other polymers. Larch Arabinogalactan also stabilizes emulsions. It has been observed in photomicrographs of oil-in-water systems containing Larch Arabinogalactan, the oil-in-water emulsion can be characterized as having smaller and more uniform oil droplets. The ability of Larch Arabinogalactan to produce smaller, more uniform droplets tends to enhance the stability of Larch Arabinogalactan-containing systems over time and is generally known to enhance performance properties. These emulsions have application in cosmetic, personal care, food and agriculture applications.

Often implicated in a tree's resistance to disease and microbial attack, terpenes concentration increases following intrusions by predators or parasitic organisms. In conifers for instance, oleoresin, a complex mixture of terpenes [FIG. 4] that includes monoterpenes, sesquiterpenes, diterpenes, triterpenes and their derivatives is an important defense strategy against bark beetles and their associated fungal pathogens [21]. This phenomenon forms the basis for ecological interactions of forest trees. High concentrations of terpenes exhibit toxic effects and play a protective role against pathogens and herbivorous animals. Some of those compounds such as volatile monoterpenes are involved in chemo-recognition, act as attractants or deterrents, and often determine the particular “bouquet” of plant material. Along with sesquiterpenes, they form the main constituents of essential oils and of oleoresins volatile fractions. Numerous studies have attributed the following properties to terpenes: antimicrobial [22,23], fungicidal [24], antiviral [25], anti-inflammatory [26], cytotoxic [27]. Inducible resin sesquiterpenoids could function as phytoalexins. In addition, the many ecological concepts that posit terpenes as mediators of plant-pathogen and plant-insect interactions can now be experimentally evaluated by recombinant approaches, which may in turn lead to new strategies for crop protection. Biotechnological applications made possible by recent molecular advances include the engineering of terpenes-based defenses in crop plants.

Worldwide demand for bioactive molecules of natural origin has progressed sharply in recent years, due to several factors such as new consumer awareness, cultural and societal changes as well as expanded knowledge in energy alternatives and natural raw materials. Increased exposure to “green” trends in the media and in wider distribution networks also contributed to higher growth in those sectors. The continued economic toll taken by microbial pathogens suggests a need to develop new, more effective approaches for preventing microbial infection, particularly fungal infection of seed crops, such as barley, wheat, corn, and rice. Additionally, these requirements should be met without significant adverse side effects to the plant or environment, and without seriously restricting planting or growth conditions, or requiring expensive chemical treatment of either growing plants or harvested seeds.

Microbial infection of monocot seeds, meaning seed infection due to a fungal, bacterial or viral agent, is a significant agricultural problem, often resulting in pronounced loss of seed quality and usability. Monocot seeds are susceptible to pathogen, e.g., fungal infection, both in the field and as stored seeds. Although some fungi infect seeds under both field and storage conditions, the main genera of fungi falling into these two classes is divided. Field fungi invade seeds during their development on plants in the field or following harvesting while the plants are standing in the field. Field fungi require a high moisture content for growth. Thus, periods of high rainfall at harvest can result in extensive grain deterioration. The main fungal species associated with crops such as wheat or barley in the field are Alternaria, Fusarium, and Helminthosporium species, while Fusariummoniliforme primarily attacks corn. Other field fungi associated with grain crops include Cladosporium and Trichoderma ssp. Species of storage fungi that may be present on the seeds and which develop during wet storage are mostly, although not strictly, of the genera Aspergillus and Penicillium, and infest seeds under storage or germination conditions. Some of the major deleterious effects of storage fungi on seeds are to (i) decrease viability, (ii) cause discoloration, (iii) produce mycotoxins, (iv) cause heat production, and (v) develop mustiness and caking. To fight this problem, producers need to use diverse agrochemical products in order to guarantee a good development of their future crops. Nevertheless, now the world market tendency is to be increasingly more cautious to accept the use of pesticides or fungicides on crops. Simultaneously, each year the world demand for healthy, pesticide-free and chemical-free food, as fruits and vegetables, is growing. Therefore, it would be very advantageous to have a product that naturally protects crops against these pathogens. Sunburn is generated while fruit is still on the tree and is exposed to particularly high amounts of solar radiation and high temperatures. Various degrees of sunburn can be distinguished on fruit, ranging from a slight discoloration of the natural fruit pigment to a severe burn that completely destroys (ulcerates) the plant tissue, in the worst case. In order to prevent or decrease sunburn or sun damage, plants, plant parts, fruits and/or flowers are sought to be protected against the harmful effects of heat and excessive UV radiation. Therefore, it would be very advantageous to have a product to reflector screen said radiation excess, or products that can absorb said radiation excess. Fire blight, a devastating bacterial disease in pome fruits, causes severe economic losses worldwide. Hitherto, an effective control could only be achieved by using antibiotics, but this implies potential risks for human health, livestock and environment. Whereas fungal diseases can be controlled by use of modern fungicides, control of bacterial diseases is much more difficult. In pome fruit trees, the prevailing cultivars are particularly susceptible to the enterobacterium Erwiniaamylovora, the causal agent of fire blight. Despite all efforts of restriction, there is a progressive spread of the disease. E. amylovora may infect flowers early in the season and later invades shoots (primary and secondary fire blight). The disease threatens developing fruits and may also affect fruit load of the following years by killing fruit spurs, branches and, even worse, the whole tree. Resistant cultivars, even if available, do not completely fit commercial demands with respect to fruit quality, yield or other agronomic features. The only effective chemical control is achieved by the use of antibiotics, such as streptomycin. Due to the general concerns against the use of antibiotics as crop protectants, alternative and toxicologically as well as environmentally safe strategies are urgently needed. A new strategy, particularly useful for the control of secondary tire blight, involves the use of the plant growth regulators. Plant growth regulators (PGR's) are those substances used for controlling or modifying plant growth processes without severe phytotoxicity. For best results, PGRs should be handled as production tools, like water and fertilizer. PGRs can be an integrated part of crop production cycle. They are most effective when applied at the appropriate times to regulate plant growth or development. In other words, growth retardants cannot “shrink” an overgrown plant. They must be applied before the plant is overgrown to avoid plant stretch. Plant growth regulators are classified as pesticides. PGR's are not conventional pesticides. They are, however, are subject to all of the same regulatory authorities recordkeeping and rules as all of other pesticides. Their use is governed by the manufacturer's label as with other pesticides. They are used to control “any injurious or troublesome organic function of a plant.” Therefore, it would be very advantageous to have a product that achieves above mentioned targets but the product is not antibiotic however is alternative and toxicologically as well as environmentally safe.

US 2007/0232495 A1 discloses the compositions and methods that naturally protect plant tissues against ultraviolet radiation and temperature, thus giving protection against sunburn to plants, plant parts, fruits and/or flowers during their development by adding phenolic acids and, more specifically, cinnamic acid derivatives, among which are p-coumaric acid, ferulic acid, caffeic acid and sinapic acid [33].

WO/1998/059056 discloses intermediate Secondary Metabolites in practicing the method of the invention, there is first identified an intermediate secondary metabolite that can be shown to inhibit infection of a plant pathogen in monocot seeds, at above-normal levels of the metabolite. Pathonen-Resistant Mutants Plant cells or plant tissue, and typically seeds, from mutated plants were tested for the ability to resist infection by a selected microbial pathogen, e.g., a fungal pathogen such as Alternaria, Fusarium, Helminthosporium, F. moniliforme, Cladosporium or Trichoderma ssp. Plant tissue, e.g., seeds, identified as inhibiting growth of the pathogen were then further analyzed to identify (i) an intermediate secondary metabolite present in the seeds at elevated levels, and (ii) the phenotypic change in the plant cell that caused the increase in levels of the secondary metabolite, toxic to the pathogen [34].

Present invention is directed to the use of larch wood extracts as natural compounds in particular antioxidant Dihydroquercetin (taxifolin), polysaccharide Larch Arabinogalactan, Larch Arabinogalactan combined with Dihydroquercetin (taxifolin) and wood oleoresin comprising oil and resin for applications in agriculture, wherein wood extracts are suggested to use as natural compounds for exposing numerous benefits within commercial agricultural purposes, that play protective role against external aggressions may be caused by UV radiation, free radical generation, hydric stress, attack of pathogenic agents, herbivorous animals attack, as well as these natural compounds are bioactive molecules with specific and nonspecific effects on intra and extra organismal plant signaling mechanisms.

### SUMMARY OF THE INVENTION

The present invention addresses some of the above issues. In one aspect of the invention, the wood extract in particular Dihydroquercetin (taxifolin) is provided for reducing or inhibiting free radical oxidative damage, harmful pathogen and fungi effects, for protection of plant tissues against ultraviolet radiation and temperature, thus giving protection against sunburn damage, for increasing adaptive potential of the seedlings, increasing the root growth of seedlings, to maximize the ability of plants to grow vigorously in response to changing environmental conditions, thus agricultural compositions may include an effective amount of Dihydroquercetin (taxifolin) extract. The wood extract may be standardized to about 80% Dihydroquercetin (taxifolin).

Another aspect of the invention includes the use of larch wood extract as natural compound that contain the combination of a Dihydroquercetin (taxifolin) and Larch Arabinogalactan as one extract to reduce or inhibit free radical oxidative damage, harmful pathogen and fungi effects, to protect plant tissues against ultraviolet radiation and temperature, thus giving protection against sunburn damage, to increase adaptive potential of the seedlings, to increase the root growth of seedlings, to maximize the ability of plants to grow vigorously in response to changing environmental conditions resulting in agriculture benefits. In one aspect, the polysaccharide Arabinogalactan can be defined as a fiber containing significant amounts of natural antioxidants, mainly Dihydroquercetin (taxifolin) associated naturally to the fiber matrix with the following specific characteristics: 1. Fiber content, higher than 70% dry matter basis. 2. The flavonoid Dihydroquercetin (taxifolin) must be an intrinsic properties as described in present invention, derived from natural constituents of the material not by added Dihydroquercetin (taxifolin) or by previous chemical or enzymatic treatments. It has been found that surprisingly and unexpectedly the combination of Larch Arabinogalactan and Dihydroquercetin (taxifolin) synergistically can effectively be used for agriculture applications and serve as a natural pool of nutrients and growth factors that support plants, plant parts, fruits and/or flowers against many of the stressors e.g. to be the second line of defense thus agricultural compositions may include an effective amount of Larch Arabinogalactan combining with Dihydroquercetin (taxifolin).

Another aspect of the invention includes the use of larch wood extract as natural compound that contain wood oleoresin extract in forms of oil and/or resin an another class of natural actives that supports plants, plant parts, fruits and/or flowers from different stress conditions in particular harmful pathogens effects, the extract comprising neutral part of oil and resin especially derived from wood oleoresin tbor application in agriculture preparations or formulations in order to provide active role in limiting pathogens effects, to provide effective early defense in response to pathogens, to increase adaptive potential of the seedlings, to increase the root growth of seedlings, to maximize the ability of plants to grow vigorously in response to changing environmental conditions resulting in agriculture benefits thus agricultural compositions may include an effective amount of wood oleoresin extract. In one aspect, the oleoresin can be combined with Dihydroquercetin (taxifolin) naturally as one extract or syntactically, wherein Dihydroquercetin (taxifolin) might be in content of 0.1% to 30%. In another aspect, the oleoresin can be combined with Larch Arabinogalactan combining naturally with Dihydroquercetin (taxifolin), wherein Larch Arabinogalactan combining naturally with Dihydroquercetin (taxifolin) might be in content of 1% to 80% of oleoresin, particularly larch oil.

For the purposes of the present invention, the terms preparation and formulation are also used synonymously in addition to the term composition.

The wood extracts or natural compounds and/or their derivatives and/or mixture of natural compounds and their derivatives may be applied to the agriculture composition by mixing them in preparation so that natural compounds are retained with the composition in an amount effective to achieve above objects or purposes of present invention. Alternatively, the natural compounds and/or their derivatives and/or mixture of natural compounds and their derivatives can be applied using a technique selected from the group consisting of spraying, dipping, rinsing, brushing, or a combination thereof.

It is to be understood that this invention is not limited to the particular compositions, methodology, or protocols described herein. Further, unless defined otherwise, all technical and scientific terms used herein have the same meaning as commonly understood to one of ordinary skill in the art to which this invention belongs. It is also to be understood that the terminology used herein is for the purpose of describing particular embodiments only, and is not intended to limit the scope of the present invention, which will be limited only by the claims.

It is to be understood that, unless otherwise specifically noted, all percentages recited in this specification are by weight.

### BRIEF DESCRIPTION OF THE DRAWINGS

The present invention is further illustrated by reference to the accompanying drawing, in which:

FIG. 1 depicts steric structure of Dihydroquercetin (taxifolin) molecule;

FIG. 2 depicts Larch Arabinogalactan molecule structure;

FIG. 3 depicts Larch Arabinogalactan component units;

FIG. 4 depicts organization of terpene biosynthesis in plants; and The pathways of monoterpene, sesquiterpene, and diterpene biosynthesis are conveniently divided into several stages. The first encompasses the synthesis of isopentenyldiphosphate, isomerization to dimethylallyldiphosphate, prenyltransferase-catalyzed condensation of these two C5-units to geranyldiphosphate (GDP), and the subsequent 19-4 additions of isopentenyldiphosphate to generate farnesyl (FDP) and geranylgeranyl (GGDP) diphosphate[28]. In the second stage, the prenyldiphosphates undergo a range of cyclizations based on variations on the same mechanistic theme to produce the parent skeletons of each class. Thus. GDP (C10) gives rise to monoterpenes [29]. FDP (C15) to sesquiterpenes [30], and GGDP (C20) to diterpenes [31]. These transformations catalyzed by the terpenoid synthases (cyclases) may be followed by a variety of redox modifications of the parent skeletal types to produce the many thousands of different terpenoid metabolites of the essential oils, turpentines, and resins of plant origin [32].

FIG. 5 depicts flavonoid biosynthetic pathway. Naringenin is a central metabolite of the flavonoid biosynthesis pathway, and is synthesized upstream of the first cytochrome P450 hydroxylation event in the pathway (F3′H). As such, naringenin was chosen as a P450-precursor control for chemical complementation attempts. Dihydroquercetin (taxifolin), a product of the first P450 hydroxylation was chosen as an experimental compound to determine if its addition to the growth media could rescue the mutant phenotype of cyp705a5.

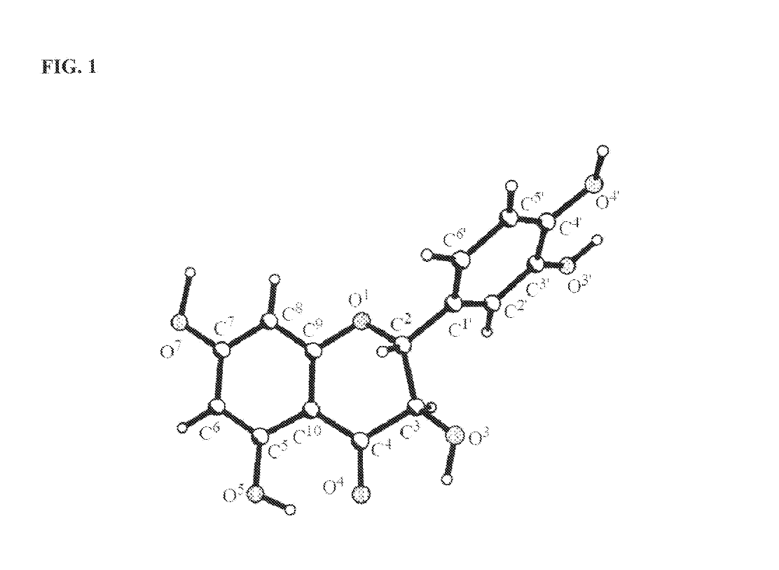

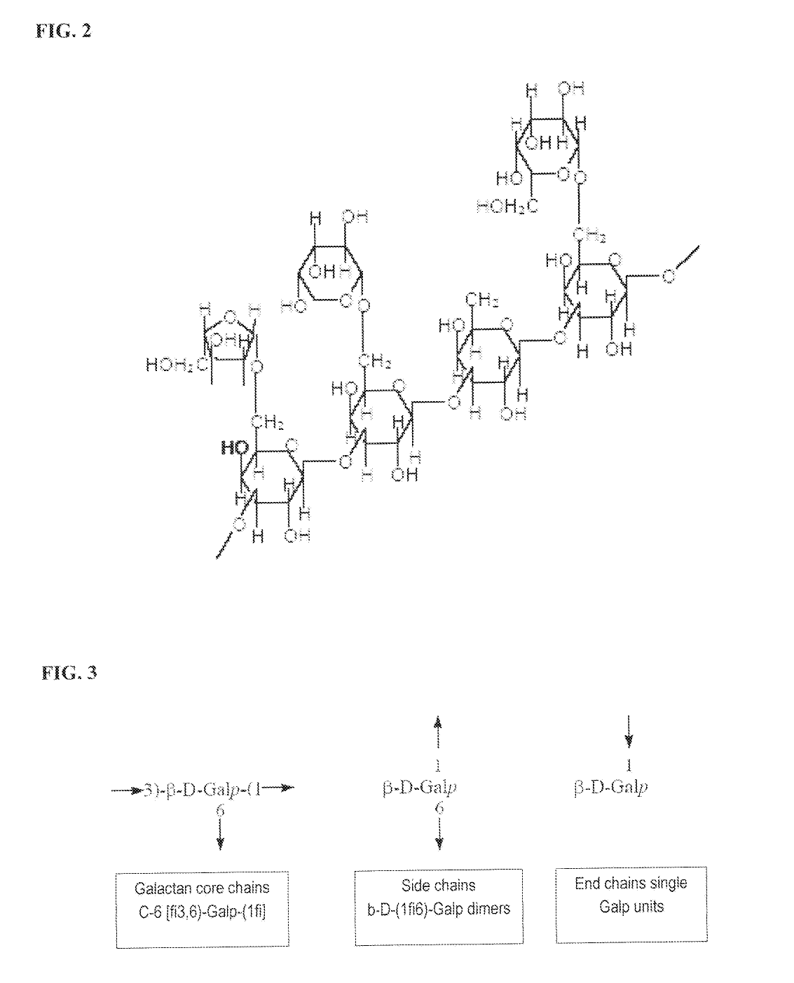

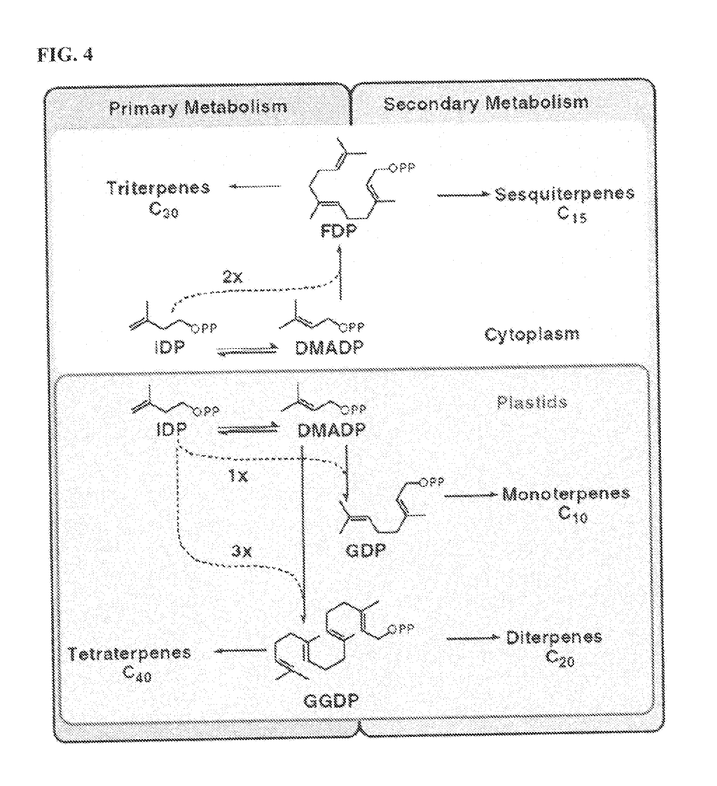

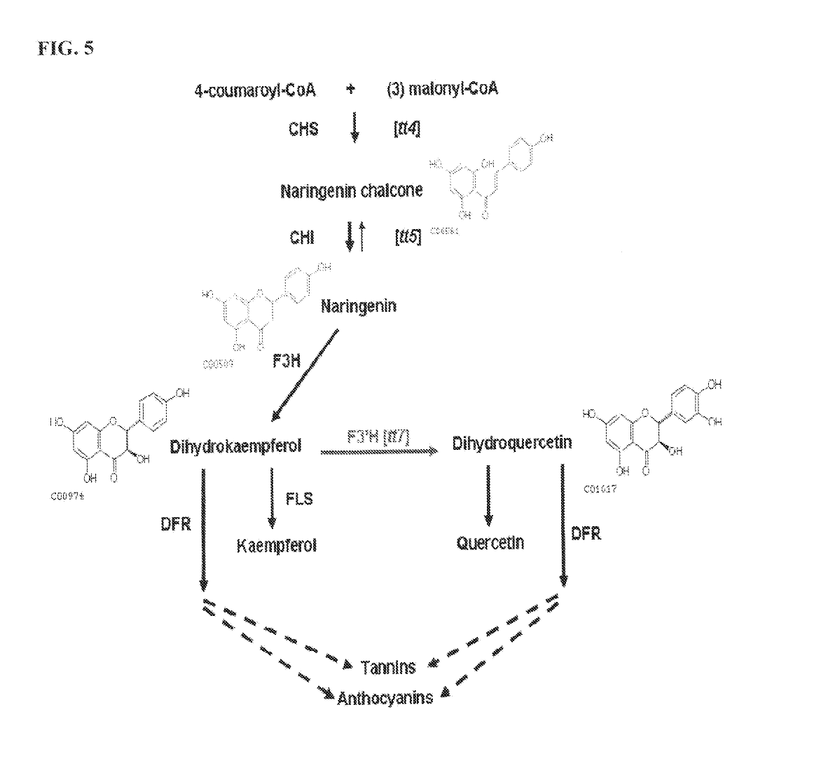

### DETAILED DESCRIPTION OF THE INVENTION

Before the present composition and methods of use are disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and is not intended to be limiting since the scope of the present invention will be limited only by the appended claims and equivalents thereof.

In this invention, we have found that it is useful to apply larch wood extracts with the purposes to give more resistance to plants, plant parts, fruits and/or flowers against pathogens as bacteria and fungi, to protect plant tissues against ultraviolet radiation and temperature, thus giving protection against sunburn to plants, plant parts, fruits and/or flowers during their development, to increase adaptive potential of the seedlings, increase the root growth of seedlings, to maximize the ability of plants to grow vigorously in response to changing environmental conditions.

According to the present invention “larch wood extracts” refers to all kind of extractable raw wood material obtained from a tree of the genus Larix. Preferably the wood extracts are obtained from extractable raw wood material used from Larixcajanderi, Larixczekanowskii, Larixdahurica, Larixgmelinii, Larixkamtschatica, Larixrussica, Larixsibirica, Larixsukaczewii. The larch wood material can, however, derive from other members of the genus Larix as well. Preferably the extractable larch wood material is larch sawdust, which is a waste product in wood industry. It is inexpensive and easily available in high amounts. The term “larch sawdust” also refers to larch wood shavings. Other kinds of waste wood from larch (e.g. bark, wastes accruing in woodcutting, scrap wood) can also be used within the frame of the present invention.

The present invention provides the use of wood extracts comprising an effective amount of Dihydroquercetin (taxifolin) and Larch Arabinogalactan. In a preferred embodiment, this Larch Arabinogalactan is non-starch soluble polysaccharide defined as dietary fiber, emulsifier, stabilizer, binder or bonding agent in essential oils, humectants, non-nutritive sweetener, flavor base, processing aid and stabilizer and considered to be bioavailability-enhancing and surface active agent, which can function as surfactant, emulsifier, foam modulator, and/or active ingredient dispersion agent, which is extracted mainly from plant genus Larix, especially from the Larixcajanderi, Larixczekanowskii, Larixdahurica, Larixgmelinii, Larixkamtschatica, Larixrussica, Larixsibirica, Larixsukaczewii. In another preferred embodiment, the Dihydroquercetin (taxifolin) is flavonoid defined as antioxidant, anti-inflammatory, anti-pathogen, anti-microbial, photo protection and plant growth stimulator compound, which is extracted mainly from plant genus Larix as described above. In a third preferred embodiment the use of larch wood extracts comprises effective amounts of Larch Arabinogalactan combined with Dihydroquercetin (taxifolin) defined as a dietary fiber containing significant amounts of natural antioxidants associated naturally to the fiber matrix, with the following specific characteristics: 1. Fiber content, higher than 70% dry matter basis. 2. The flavonoid Dihydroquercetin (taxifolin) must be an intrinsic properties, derived from natural constituents of the material not by added Dihydroquercetin (taxifolin) or by previous chemical or enzymatic treatments, wherein Larch Arabinogalactan combined with Dihydroquercetin (taxifolin) extracted mainly from plant genus Larix as described above. In a final preferred embodiment, the larch wood oleoresin extract in forms of oil and/or resin defined as the class of natural actives that supports plants, plant parts, fruits and/or flowers from different stress conditions in particular harmful pathogens effects and provides effective early defense in response to pathogens, increases adaptive potential of the seedlings and root growth of seedlings, maximizes the ability of plants to grow vigorously, which is extracted mainly from plant genus Larix as described above, wherein the oleoresin can be combined with Dihydroquercetin (taxifolin) naturally as one extract or syntactically, wherein Dihydroquercetin (taxifolin) might be in content of 0.1% to 30% and wherein the oleoresin can be combined with Larch Arabinogalactan combining naturally with Dihydroquercetin (taxifolin), wherein Larch Arabinogalactan combining naturally with Dihydroquercetin (taxifolin) might be in content of 1% to 80% of oleoresin, particularly larch oil.

In the context of this invention, the above described larch wood extracts can be prepared with suitable carriers, and which larch wood extracts preparation is provided in a form that is suitable for being used in agricultural purposes, agrochemical application, preparations, formulations and compositions. The agrochemical preparations of the invention may be prepared in accordance with methods known in the art and may be in the form of a dry or a liquid preparation.

In another specific embodiment, the agrochemical preparation and\or composition of the invention is a stabilized liquid composition, which may be an aqueous or oil-based slurry.

Larch wood extracts, particularly flavonoid Dihydroquercetin (taxifolin) or polysaccharide Arabinogalactan or Arabinogalactan combining with Dihydroquercetin (taxifolin) or wood oleoresin comprising oil or resin or the mixture of above ingredients thereof may be incorporated into agrochemical compositions in an amount from about 0.1% to about 50%, or in an amount from about 0.1% to about 30%.

Further preferred combinations of embodiments are disclosed in the claims.

In many host plants, resistance against a wide range of fungi and bacteria is connected to the presence of phenolic compounds, in particular flavonoids [35,36]. The flavonoids are a large class of plant secondary metabolites. They contain a 15-carbon phenylpropanoid core, which is extensively modified by rearrangement, alkylation, oxidation, and glycosylation. In plants the flavonoids fulfill a diverse array of roles including pigmentation and protection against UV photodamage and can act as signaling molecules [37]. In addition, these compounds are involved in photosensitization and energy transfer, the actions of plant growth hormones and growth regulators, the control of respiration, photosynthesis, morphogenesis, and sex determination, as well as defense against infection. During the last few decades, a flavonoid Dihydroquercetin (taxifolin) has been extensively studied due to its wide spectrum of unique biological properties. Dihydroquercetin (taxufolin) was discovered as a part of phenolic complexes derived from numerous plants. Dihydroquercetin (taxifolin) relates to the class of dihydroflovonols in the flavonoid biosynthesis corresponding to Flavanone 3-hydroxylase (FHT or F3H), a 2-oxoglutarate dependent dioxygenase catalyzing the hydroxylation of flavanones (eriodictyol, kaempferol, mericitin) to dihydroflavonols (dihydroquercetin, dihydrokaempferol, dihydromericitin) [38].

Intermediate secondary metabolite Dihydroquercetin (taxifolin) is a secondary metabolite that is further converted by plant enzymes (DHQR) to another secondary metabolite. DFR and DHQR are synonymous, and refer to the enzyme dihydroflavanolreductase (DFR), also referred to as dihydroflavanol-4-reductase, also known as dihydroquercetinreductase (DHQR). In barley, formation of proanthocyanidins and anthocyanidins is controlled by the Ant genes [39]. The enzymes involved in the formation of procyanidin B-3 (a dimer of (+)-catechin) and prodelphinidin B-3 (a dimer of (+)-gallocatechin) in barley. Ant18 is the Hordeumvulgare (barley) structural gene encoding dihydroflavonolreductase (DFR), also referred to as dihydroquercetinreductase (DHQR) [40]. The single copy Ant18 gene encodes DHQR, which catalyzes the last common step in the flavonoid biosynthetic pathway leading to anthocyanins and proanthocyanidins. Structural genes encoding DHQR have been cloned in other plant species including Z. mays, Antirrhinum majus, and P. hybrida (40). In an NADPH-dependent reaction, this enzyme reduces dihydroquercetin to 3,4-cis-leucoanthocyanidins. Mutant plants containing these ant18 alleles lack DIHQR activity. For the mutant alleles ant18-159, ant18-162 and ant18-164, the levels of DHQR-specific transcripts are similar to wild-type plants [40]. Messenger RNA for DH-IQR is not detectable in ant18-164 cells, and has been attributed to mutations in the pre-mRNA splice donor site [40]. Studies conducted in support of the present statement have shown that certain mutations in the Ant18 gene (i) were effective to provide an accumulation of dihydroquercetin in the testalpericarp, and (ii) resulted in the formation of seeds that were extremely resistant to attack by Fusarium. While the wild types accumulate about 130 mg catechin and proanthocyanidins (downstream products) per 100 g dry weight, the ant18 mutants (e.g., ant18-102, -141, -159, -186) contain about 0.60 to 0.70 mg dihydroquercetin per 100 g dry weight [41]. However, this small amount of dihydroquercetin accumulated in the testa of ant18 mutants is effective to provide an extraordinary resistance to Fusarium infection. Accumulation of dihydroquercetin in the outer seed layers imparts Fusarium resistance in the mutant. Since buildup of dihydroquercetin by lack of enzymatically active DHQR or defective DHQR transcript maturation has been shown herein to confer seed resistance to Fusarium infection, antisense inhibition of the gene encoding DHQR in testa cells will lead to the accumulation of dihydroquercetin. Set of genes encoding key enzymes involved in the synthesis of secondary metabolites derived from several branches of the soybean phenylpropanoid pathway in response to Psg infection.

Barley (Hordeumvulgare L.) seeds were soaked in aqueous 10-4 M dihydroquercetin (DHQ) or dihydroquercetin (taxifolin) to examine its influence on seed germination and further growth of seedlings under optimal soil watering and flooding conditions. The adaptive potential of the plants was estimated by the content of thiobarbituric acid reactive substances (TBARs) and the activity of ascorbate peroxidase (AsP). High-grade seeds were germinated evenly under (−DHQ)- and (+DHQ)-treatments. Low-grade seeds soaked in DIHQ, showed no mold and twofold germination rate in comparison with the same seeds soaked in water. The seedlings grown from the similarly germinated seeds did not differ from each other in the shoot growth, independent of the DHQ-pretreatment. The root growth was higher in DHQ-pretreated plants. Soil flooding suppressed the shoot and root growth rates in non-pretreated and DHQ-pretreated plants, however TBARs content was lower in the roots and leaves of (+DHQ)-seedlings as compared to the (−DIHQ)-ones. The activity of AsP increased more significantly in the (+DHQ)-plants. The ratio between TBARs content and the AsP activity was lower in the leaves of (+DHQ)-plants both under optimal soil conditions and flooding. Thus, the treatment of low-grade barley seeds with DHQ protects the seeds against mold and increases adaptive potential of the seedlings [42].

Dihydroquercetin (taxifolin) is identified to be an intermediate secondary metabolite that can be shown to inhibit infection of a plant pathogen in monocot seeds, at above-normal levels of the metabolite. Microorganisms that have been associated with monocots during or after the malting stage include Aureobacterium, Cladosporium, Mucor, Penicillium, Fusarium, Aureobasidiumpullulans, Trichosporon. Alt. alternata, Rhizopus. Candida. Cryptococcus, Richia, Rhodotorula, Pseudomonas, and Bacillus (Flannigan, 1996). Proposal is based on the discovery that the buildup of certain secondary intermediate metabolites in seeds is effective to block microbial, e.g., fungal, infection of the seeds. By placing under the control of a seed-inducible promoter, an inhibitory gene sequence whose expression results in a buildup of the metabolite in seeds (structural gene sequence), it is possible to specifically induce pathogen resistance in seeds, or within certain seed tissues, such as the seed testa layer, without significantly interfering with plant growth and development and other phenotypic traits in the plant as a whole. That is, the transformed monocot plants produce a secondary metabolite at levels toxic to a given pathogen in seeds or localized seed tissue only, allowing the plant itself to develop normally and at normal seed production levels. The use of natural compounds has been proposed as a possible alternative to chemical fungicides. For example in one Italian study phytoalexins, such as dihydroquercetin, umbelliferone, ferulic acid, esculetin, scopoletin, scoparone, 6-methoxymellein and resveratrol, were tested to control P. expansum (the agent of blue mold, causes considerable postharvest losses of apples and other pome fruits, a pathogen which is of health significance, since it produces patulin, a mycotoxin with genotoxic properties, known to cause immunological, neurological and gastrointestinal toxic effects in animal models) and patulin production in in vivo and in vitro trials. In in vivo trials surface sterilized apples (variety Golden Delicious) were wounded, treated with each phytoalexin, inoculated with a toxigenic strain of P. expansum and incubated at 16° C. in the dark. Lesion diameters were recorded every 2 days. After 14 days, tissue samples from untreated and treated lesions were collected and analyzed by HPLC for their patulin content. In in vitro trials fungal growth and patulin production were evaluated on agarized apple juice medium. Among the tested phytoalexins, umbelliferone and dihydroquercetin were the most effective in reducing fungal infections in apples, although no reduction in patulin accumulation was observed. In particular, umbelliferone reduced lesion diameters by 100%, 68% and 38% and dihydroquercetin by 97%, 71% and 27% after 6, 10 and 14 days of incubation, respectively. When tested in vitro, umbelliferone and dihydroquercetin reduced patulin accumulation by 53% and 48%, respectively, although no inhibition of fungal growth was observed. These results suggest that the control activity of umbelliferone and dihydroquercetin towards P. expansum may be related to their ability to enhance defence responses in the host[43].

In another study the purpose was to determine the effects which dihydroquercetin (DHQ) has on the natural healing process of suberization and wound periderm initiation in cut seed potatoes. Anatomical studies of treated seed potato tissue showed that treatment with DHQ increases the thickness of the suberized layer formed below the cut surface. The fungal mold growth on treated seed pieces incubated in a controlled environment was adequately retarded. The longer the treatment material was allowed to remain on the seed piece, the greater was the protection against mold growth afforded by the mixture [44].

Flavonoids also have apparent roles in plant stress defense, such as in protection against damage caused by pathogen attack, in wounding or in excess of UV light. The low availability of nitrogen or phosphorus, and low temperatures affect flavonoid levels in plants [46,47]. Flavonoids, mainly anthocyanins are responsible for the autumn colors in many plant species; they have been suggested to protect leaf cells from photo-oxidative damage, thereby enhancing the efficiency of nutrient retrieval during senescence [47]. The localization of flavonoids in the epidermal layers of plants and their known ultraviolet absorptive properties has led to a suggestion that they can serve as shields against potentially harmful radiation. There is a growing body of evidence for the role of flavonoids in photoprotection. A variety grape cell cultures produce anthocyanins and exhibit a red appearance, although the major cell population is composed of non pigmented cells. The addition of dihydroquercetin (DHQ) resulted in a 10-fold increase in dihydrotlavonol 4-reductase (DFR) activity. In non pigmented cells a higher level of precursors or an elevated level of substrate availability would induce anthocyanin synthesis [48].

Vacuoles of the plants have various physiological functions in plant development and differentiation. One of their functions is to act as a storage site for various secondary metabolites including flavonoids. The pH gradient between the cytosol and the vacuole provides the potential energy needed to take up substrates into the vacuole. It was suggested that vacuolar uptake of flavonoids was dependent on the energy provided by the proton gradient (H+). Hence, in vivo the fundamental energy transduction machinery of cells runs mainly on H+ gradients and proton-coupled electron transfer reactions. For instance, regulation of intracellular pH is so central to the living state that even the most primitive cells capable of energy transduction must have been able to control intracellular pH by expelling H+(proton pump) produced from the hydrolysis of organic compounds [49]. The effect of dihydroquercetin (DHQ) on proton pumps of the vacuolar membrane (H+-ATPase and H+-pyrophosphatase), slow vacuolar (SV) channel, lipid peroxidation, and stability of isolated vacuoles was studied. The results of experiments showed that DHQ affected active and passive transport systems of the vacuolar membrane. The mechanism of action of DHQ may be based on its combined effect on the sulfhydryl groups of proteins and the lipid component of the membrane. The strong stabilizing effect of DHQ on the membranes of isolated vacuoles may be associated not only with its antioxidant properties but also with changes in the membrane permeability affecting the ion channels as the buffer [50]. For instance, pH is the concentration of hydrogen ions (H+). Buffers are molecules which take in or release ions in order to maintain the H+ ion concentration at a certain level. When environment's pH is too low and it becomes too acidic (acidosis), the presence of too many H+ ions is to blame. Buffers help to soak up those extra H+ ions. On the other hand, the lack of H ions causes the environment's to be too basic (alkalosis). In this situation, buffers release H+ ions. It is also essential for human as the buffers function to maintain the pH of our blood by either donating or grabbing H+ ions as necessary to keep the number of H+ ions floating around the blood at just the right amount.

Plant compounds of various chemical natures are involved in the regulation of physiological processes, increase the plant resistance to adverse environmental factors, and play an essential role in the plant protection against pests and diseases. First and foremost, these are phytohormones, whose role in the growth processes and defense functions is most well studied, as well as phenolic and aromatic compounds, the obligatory components of metabolic processes, whose regulatory functions are yet vague and stimulate an increased research interest worldwide. Auxins are compounds that positively influence cell enlargement, bud formation and root initiation. They also promote the production of other hormones and in conjunction with cytokinins, they control the growth of stems, roots, flowers and fruits. Auxins were the first class of growth regulators discovered. Auxin is the active ingredient in most rooting compounds in which cuttings are dipped during vegetative propagation. The most common auxin found in plants is indoleacetic acid or IAA. Numerous studies have demonstrated that plant phenols are capable of influencing the plant hormonal status and activate or inhibit certain enzymatic systems. For example, the regulatory role of phenolic compounds is connected with the control of auxin levels during seed germination, inhibition of enzymes during seed dormancy, involvement in plant defense mechanisms, tissue respiration, and enzyme biosynthesis. Because the active flavonoids are widely distributed in the plant kingdom and exert their effects at micromolar concentrations approximating likely endogenous levels, they may act as natural auxin transport regulators in plants [51]. The effect of some flavonoids on the polar transport of auxins was investigated in hypocotyl sections of dark grown seedlings of cucumber, Cucumissativus L., by means of ¹⁴C labelledauxins. In experiments of 4-6 h duration quercitrin, morin, dihydroquercetin (taxifolin), naringin, sulfuretin and ferulic acid increased the polarity of the transport of indol-3yl-acetic acid (stimulation of basipetal, inhibition of acropetal transport) [52]. Dihydroquercetin (taxifolin) increased the root growth of cress seedlings and stimulated the oxygen consumption of white potato tissue. Both effects were biphasic and clearly dose dependent and showed a high degree of reciprocal correlation. For the mechanism of action an interaction of Dihydroquercetin (taxifolin) with IAA-oxidase (auxins oxidase) was proposed [53]. Dihydroquercetin (taxifolin) increases the root growth of cress [Lepidiumsativum] seedlings and stimulates the O2 consumption of white potato [Solanumtuberosum] tissue. Both effects are biphasic and dose dependent and show a high degree of reciprocal correlation. For the mechanism of action an interaction of Dihydroquercetin (taxifolin) with IAA-oxidase was proposed [45].

A plant's response to gravity, known as gravitropism, is made up of an intricate series of genetic, biochemical, and physiological events. Plants detect positional changes and adjust their growth accordingly to regain their original orientation with respect to gravity. Developmental processes leading to the formation of specific plant architecture are influenced by gravity. Gravitropism has been separated into three major events: perception, signal transduction, and the growth response. The prevailing model for tropic growth responses indicates that directional growth is the result of the lateral redistribution of the plant growth regulator indole-3-acetic acid (IAA; auxin). Auxin has a major role in nearly every aspect of plant growth and development. Auxin is synthesized in the growing tips of vegetative shoots and is transported with distinct polarity throughout the plant via a specialized system of auxin influx and efflux protein complexes embedded in the plasma membrane. It was studied that upon addition of 15 μMDihydroquercetin (taxifolin) to the growth medium, the phenotype was restored ≧100% of the wild type gravity response. Dihydroquercetin, a product of the first P450 hydroxylation was chosen as an experimental compound to determine if its addition to the growth media could rescue the mutant phenotype of cyp705a5 [54].

Plant growth regulators (PGR's) are those substances used for controlling or modifying plant growth processes without severe phyto-toxicity. For best results, PGRs should be handled as production tools, like water and fertilizer. PGRs should be an integrated part of your crop production cycle. They are most effective when applied at the appropriate times to regulate plant growth or development. In other words, growth retardants cannot “shrink” an overgrown plant. They must be applied before the plant is overgrown to avoid plant stretch. Plant growth regulators are classified as pesticides. Plant growth regulators (PGR's) play an important role not only in solving crop biomass augmentation problem, but also in growing the plants with improved, from physiological and biochemical standpoints, characteristics for further industrial processing. Dihydroquercetin (taxifolin) based bio substance shows qualified plant growth regulator “supplement” functions to enhance root development or the vegetative by evaluating following claims which include, but are not limited to, breaking dormancy, induction of early germination or sprouting, root development and vegetative growth promotion. However, Dihydroquercetin based bio substance shows pest control product claims including modification of physiological, morphological and reproductive processes of plants other than those within the meaning of “supplement”. Such claims include, but are not limited to, the inhibition of sprouts or germination, fruit setting, sucker control, color enhancement. It were shown the results of research on the biological, economic, and energetic efficiency of the application of natural Dihydroquercetin (taxifolin) based growth regulators for growing spring rape, which promote the increase in the seed productivity 0.29 t/ha, the output of raw fat −0.15 t/ha, the raw protein −0.08 t/ha, are determined. The fungicide activity of growth regulators is also revealed, which helps to diminish the communication and development of the disease Alternariabrassicicola and positively influences the sowing qualities of spring rape seeds [55]. According to the Russian Ministry of Agriculture. Dihydroquercetin (taxifolin) is considered a growth regulator for certain crops when used at the ranges 20 mL/ton to 250 mL/ton. Specifically, the following crops are outlined in the document (page 284-285 plant growth stimulators register of Russian Ministry of Agriculture): 1. Wheat, winter crop; 2. Wheat, spring crop; 3. Barley, spring crop; 4. Potato; 5. Grapes; 6. Sunflower; 7. Long-fibred Flax; 8. Sugar-beet. Dihydroquercetin (taxifolin) is stated to increase harvest and immunity against diseases (wheat, barley, potato, grapes, sunflower, long-fibred flax, and sugar-beet), accelerate ripening (barley), increase bunch of grapes, increase level of sugar (grapes and sugar-beet), and increase oil content of sunflowers.

Displaying various biological activities—fungicide, signal, and growth regulating—coniferous metabolites are first and foremost interesting as a source of plant extracts that are able to induce resistance of agricultural plants to pathogens and adverse environmental conditions. Evergreen coniferous trees, especially those from the family of Pinaceae are considered rich sources of Dihydroquercetin (taxifolin). During 1965-1975, extensive research on phenolic compounds isolated from pine trees was conducted in Siberia and Russian Far East. The data gathered from the research indicate that different parts of trees from the Larch (Larix genus) tree species (Pinaceae family) contain significant amounts of Dihydroquercetin (taxifolin). Compositions of coniferous metabolites origin accelerate phenological stages, stimulate plant root development by 30-40%, elevate yield, and increase the resistance of agricultural plants to many pathogens [56].

The chemical and physical defense of coniferous plants (family Pinaceae) is based on their ability to synthesize oleoresin-a complex mixture of terpenoids of various structures (mono-, sesqui- and diterpenoids[57,58] and triterpenoids in some coniferous species [59] and other biologically active substances [60]. A large number of studies have been done in recent years on the antifungal activity of terpenoids of natural origin. These reports concern mainly sesquiterpenes and sesquiterpene lactones. Besides sesquiterpenes and sesquiterpene lactones, studies into other antifungal terpenoids from medicinal species also included diterpenoids and triterpenoids [61]. Resins the class of terpenes protect coniferous plants mechanically by healing over the wound site and are directly toxic to insects and microorganisms [58]. Some terpenoids are plant growth regulators, for example, isopentenyl diphosphate, sesquiterpene, and diterpenes [62]. Oleoresin (also simply termed ‘resin’ or ‘pitch’) is a viscous secretion common in coniferous trees, which is involved in the chemical and physical defense of conifers against predators (such as bettles and fungal pathogens). Oleoresin is composed in roughly equal parts of volatile turpentine (a mixture of monoterpenes and sesquiterpenes) and rosin (also known as diterpene resin acids). The exact composition of turpentine and rosin varies from one conifer species to the next, as well as depending on the resin-producing tissue. Diterpene resin acids (DRAs) are major constituents of conifer oleoresin. They play important roles in conifer defense against insects (e.g. bark boring beetle) and microbial pathogens. Diterpene acids (e.g., abietic, isopimaric, levopimaric, neoabietic and palustric acids) have been isolated from larch dahurica oleoresin and these are known from other conifers to be anti-feedants against aphids and sawflies Tannins, phenolics and terpenoids that can inhibit feeding or digestion in other insects could also affect bark beetles. The diterpene hydrocarbons in the oleoresins were present in insignificant amounts and were represented mainly by tricyclic diterpenes, among which abietadiene, dehydroabietane, and isopimaradiene were identified [63].

After the group separation of the larch wood oleoresin, 12.5% of monoterpene hydrocarbons, 0.75% of sesquiterpenes, 18% of diterpene hydrocarbons and aldehydes, 13.5% of diterpene alcohols, and 32.5% of resin acids were obtained. The qualitative and quantitative analysis of the monoterpenes established that the monoterpene fraction contained: a-pinene (20.5%), camphene (0.3%), 8-pinene (23.2%), 3-carene (49.8%), myrcene (0.3%), limonene (1.1%), and 8-phellandrene (2.3%). In the sesquiterpenoid fraction we identified 16 compounds: cyclosativene, longicyclene, alfa-longipinene, sibirene, longifolene, y-elemene, a, y and e-murolenes, beta-selinene, d-, y-, and e-cadinenes, a-humulene, calamenene, and the methyl ether of thymol, the main components being delta- and gamma-cadinenes and longifolene. From the fraction of diterpene hydrocarbons and aldehydes presented by dehydroabietane, a mixture of dehydroabietinal and abietinal and palustral. By chromatography of the diterpene alcohols were determined epimanool, larixyl acetate, and larixol. The analytical GLC of the mixture of resin acid methyl esters showed the presence in them of the esters of the acids palustric and (or) levopimaric (7.2%), isopimaric (86.2%), dehydroabietic (2.0%), abietic (4.6%), and neoabietic (traces) [64].

Application of the extraction process with base aqueous solution will open up the way to the creation of novel technology for production of natural regulators of growth and development of plants. Growth regulators like coniferous metabolites are exogenous natural organic compounds, which in very small draughts are able to influence significantly physiological process of plants growth and their development, not showing toxic effect in concentration used and not being a nutrition source. Their activity is manifested in increased crop capacity, enhanced drought- and frost-resistance of vegetative plants, as well as in better disease resistance of plants. Coniferous plants of Pinacea family produce many interesting substances, among which are those that serve as plant growth stimulators and protectors. The scientists have shown that the extract from the sum of triterpenic acids effects the morphological plants indications similar to auxin: leaves become dark-green, roots length increases and crop capacity of many cereals and forage crops raises. Tree killing bark beetles and their vectored fungal pathogens are the most destructive agents of conifer forests worldwide. Conifers defend against attack by the constitutive and inducible production of oleoresin, a complex mixture of mono-, sesqui-, and diterpenoids that accumulates at the wound site to kill invaders and both flush and seal the injury. Although toxic to the bark beetle and fungal pathogen, oleoresin also plays a central role in the chemical ecology of these boring insects, from host selection to pheromone signaling and tritrophic level interactions [65].

Higher arabinogalactan content often goes hand in hand with higher amount of flavonoid substances, in particular with Dihydroquercetin (taxifolin) [66]. Water-soluble Arabinogalactan is a typical useful surface active agent is disclosed above in the context of the bioavailability-enhancing agent that includes a solubilizing agent. Surface active agents generally are an important aspect of the agrochemical compositions, as they can function as surfactants, emulsifiers, foam modulators, and/or active ingredient dispersion agents. Their selection for compatibility with the active ingredient constituents is important. Suitable surface active agents, include those that were discussed in the context of the bioavailability/solubility enhancing agent above, are those which are reasonably stable and foam throughout a wide pH range. Arabinogalactan has a number of benefits as compared with other polysaccharide polymers. Arabinogalactan is water-soluble, occurs naturally with a narrow molecular weight distribution. While not wishing to be bound by any particular theory, it is believed that because Arabinogalactan is highly branched it is not subject to viscosity problems, as compared to other polymers. Arabinogalactan also stabilizes emulsions. It has been observed in photomicrographs of oil-in-water systems containing Arabinogalactan, the oil-in-water emulsion can be characterized as having smaller and more uniform oil droplets. The ability of Arabinogalactan to produce smaller, more uniform droplets tends to enhance the stability of Arabinogalactan-containing systems over time and is generally known to enhance performance properties. These emulsions have application in cosmetic, personal care, food, agriculture and industrial applications. Arabinogalactan has been shown to improve dispersion of inorganic sunscreen particles leading to a more uniform and effective transference onto the surface. This leads to less clumping of the sunscreen particles and therefore more efficient packing of the sun protection per UV level. The surface tension of the gum in water depends greatly upon the purity. Pure larch arabinogalactan has a surface tension of 72.3 dynes/cm at 200 C and 10% concentration by the du Nuovy method. Technical gum under same the conditions has a value of 65.1 dynes/cm. At 30% concentration, the value of pure polymer is 71.7 dynes/cm, where is for technical polymer it is 59.9 dynes/cm. Larch arabinogalactan reacts with acid anhydrides or acyl halides to produce esters. Acetate, sulphate, propionate and benzoate esters have been made. Methyl and benzyl ethers have also been prepared. The esters and ethers are not water soluble but become soluble in moderately polar solvents, which make Arabinogalactan useful in water-in-oil agrochemical compositions.

The advantages of the present invention over pre-existing methods appears clearly from the previous descriptions and embodiments. The present invention describes new original larch wood extracts, particularly extracts from Larixcajanderi, Larixczekanowskii, Larixdahurica, Larixgmelinii, Larixkamtschatica, Larixrussica, Larixsibirica, Larixsukaczewii species. New types of agrochemical products are constantly being developed, and new raw materials are adding to the agriculture chemist's selection of ingredients in particularly larch wood extracts Dihydroquercetin (taxifolin), polysaccharide Arabinogalactan, Arabinogalactan combining with Dihydroquercetin (taxifolin), wood oleoresin comprising oil and resin. These larch wood extracts described in the present invention can easily be incorporated in a large panel of agrochemical products.

This invention has been described with reference to its preferred embodiments. Variations and modifications of the invention will be obvious to those skilled in the art from the foregoing detailed description of the invention. It is intended that all of these variations and modifications be included within the scope of the appended claims.

1. Method of using a wood extract in agriculture, comprising the steps of:
producing a wood extract from a Larix tree selected from Larixcajanderi, Larixczekanowskii, Larixdahurica, Larixgmelinii, Larixkamtschatica, Larixrussica, Larixsibirica, and Larixsukaczewii;
forming an agrochemical compositions including said wood extract; and
applying said agrochemical composition to at least one of a soil, a plant, a part of a plant, a fruit, a flower, a seedling and a propagating material.

2. The method according to claim 1, wherein the wood extract comprises a flavonoid Dihydroquercetin (Taxifolin).

3. The method according to claim 1, wherein the wood extract comprises a polysaccharide Arabinogalactan.

4. The method according to claim 1, wherein the wood extract comprises a polysaccharide Arabinogalactan and a flavonoid Dihydroquercetin (Taxifolin).

5. The method according to claim, wherein the wood extract comprises a larch oleoresin in a form of oil.

6. The method according to claim 1, wherein the wood extract comprises a larch oleoresin in a form of resin.

7. The method according to claim 1, wherein the wood extract comprises a larch oleoresin and a Dihydroquercetin (taxifolin).

8. The method according to claim 1, wherein the wood extract comprises a larch oleoresin, a Larch Arabinogalactan, and a Dihydroquercetin (taxifolin).

9. The method according to claim 1, wherein the wood extract comprises a larch oleoresin and a Larch Arabinogalactan.

10-11. (canceled)
